(19) United States
(12) Patent Application Publication
(10) Pub. No.: US 2025/0108145 A1
(43) Pub. Date: Apr. 3, 2025

(54) PUTTY FORMULATION COMPRISING MACROPOROUS HYDROXYAPATITE COMPOSITION AND METHODS OF MAKING SUCH

(71) Applicant: CAVIX AB, Uppsala (SE)

(72) Inventors: Michael Wayne Trevino PUJARI-PALMER, Uppsala (SE); Håkan ENGQVIST, Uppsala (SE)

(21) Appl. No.: 18/834,248

(22) PCT Filed: Feb. 15, 2023
(86) PCT No.: PCT/SE2023/050131

§ 371 Date: Jul. 30, 2024

(30) Foreign Application Priority Data
Feb. 16, 2022 (SE) 2250155-5

(51) Int. Cl. A61L 27/12; A61L 24/00; A61L 24/02; A61L 24/04; A61L 24/08; A61L 27/56; A61L 27/58

(52) U.S. Cl. CPC A61L 27/12; A61L 24/0036; A61L 24/0042; A61L 24/02; A61L 24/046; A61L 24/08; A61L 27/56; A61L 27/58; A61L 2400/06; A61L 2430/02

(57) ABSTRACT

The present invention relates to a putty formulation suitable for use as a bone void filler. The putty formulation comprises granules comprising a macroporous cement composition comprising hydroxyapatite and D-calcium pyrophosphate. The porosity of the composition is 60-80 vol %, the average particle size of the granules is 50-800 m, and the portion of granules in the putty formulation is 25-50 wt %.

### FIELD OF THE INVENTION

The present invention relates to a putty formulation comprising macroporous hydroxyapatite and methods of making such. The putty formulation is suitable for use as a bone void filler.

### BACKGROUND OF THE INVENTION

The interest in bone implants, such as bone void fillings, have been a hot topic for researchers during several years. Calcium phosphate-based cement (CPC) materials are considered ideal for bone replacement since they resemble the mineral phase of bone. Calcium phosphate-based cement materials are both biocompatible and biodegradable, i.e., degrade with time and is replaced with new healthy tissue, both of which are important properties for bone implants.

The aim of a bone void filling material is to have a fast resorption rate, mirroring an equally fast formation of new bone. The bone void filling material should ideally work as a template for new bone formation and prevent the formation of fibrotic tissue within the bone void. The presence of pores in a bone void filling material help to increase the bone ingrowth, which decreases the risk for implant failure.

A bone void filler is typically delivered in the form of a putty or similar that can be injected into the bone void.

WO 2015/162597 discloses a method for making a porous, chemically bonded ceramic shaped article. A porous, chemically bonded ceramic shaped article having interconnected pores, a total porosity of at least about 50%, and a microporosity of at least about 30% can be formed by such methods.

‘Fabrication of microporous cement scaffolds using PEG particles: In vitro evaluation with induced pluripotent stem cell-derived mesenchymal progenitors’ by M. Sladkova et al, Materials Science and Engineering 69 (2016) 640-652 discloses macroporous calcium phosphate cements (CPS) and a fabrication method of making such. The method enables rapid, inexpensive and reproducible construction of macroporous CPC scaffolds with tunable architecture for potential use in dental and orthopedic applications.

In the prior art there is still a need for an improved injectable formulation suitable as a bone void filler.

### SUMMARY OF THE INVENTION

The object of the present invention is to provide a putty formulation suitable for use as a bone void filler.

This is achieved by the putty formulation and method as defined in the independent claims.

An aspect of the invention relates to a putty formulation comprising:
granules comprising a macroporous cement composition comprising 80-95 wt % hydroxyapatite, and β-calcium pyrophosphate, wherein the porosity of the macroporous cement composition is 60-80 vol % as determined using Archimedes method;
a liquid carrier, and
a binder material, wherein the average particle size of the granules is 50-800 μm as determined by sieving and/or scanning electron microscopy (SEM), and the portion of granules in the putty formulation is 25-50 wt %.

In one embodiment of the invention, the macroporous cement composition further comprises α-tricalcium phosphate.

In one embodiment of the invention, the macroporous cement composition comprises 80-95 wt % hydroxyapatite, 0.1-10 wt % β-calcium pyrophosphate, preferably 1-10 wt % β-calcium pyrophosphate, and <10 wt % α-tricalcium phosphate.

In one embodiment of the invention, the portion of granules in the putty formulation is 30-50 wt %, preferably 40-50 wt %.

In one embodiment of the invention, the portion of binder material in the putty formulation is 5-25 wt %, preferably 10-25 wt %, and more preferably 13-25 wt %.

In one embodiment of the invention, the binder material comprises carboxymethyl cellulose.

In one embodiment of the invention, the binder material comprises a triblock copolymer comprising a central hydrophobic block of polypropylene glycol flanked by two hydrophilic blocks of polyethylene glycol, preferably poloxamer 407 (P407).

In one embodiment of the composition, the liquid carrier is water.

In one embodiment of the invention, the putty formulation comprises 30-70 wt % liquid carrier, preferably 35-65 wt % liquid carrier.

In one embodiment of the invention, the putty formulation comprises the granules and a gel comprised of the liquid carrier and the binder material.

In one embodiment of the invention, the putty formulation further comprises an antioxidant.

In one embodiment of the invention, the antioxidant is ascorbic acid.

In one embodiment of the invention, the putty formulation is a sterilized putty formulation.

Another aspect of the invention relates to a method of manufacturing a putty formulation comprising the steps of:
obtaining granules comprising a macroporous cement composition comprising 80-95 wt % hydroxyapatite, and at least one bioactive calcium phosphate phase, wherein the porosity of the granules is 60-80 vol % as determined by Archimedes method, and the granules have an average particle size of 50-800 μm as determined by sieving and/or scanning electron microscopy (SEM); and
mixing the granules with a liquid carrier and a binder material, wherein the portion of granules in the putty formulation is 25-50 wt %.

In one embodiment of the invention, the at least one bioactive calcium phosphate phase comprises β-calcium pyrophosphate.

In one embodiment of the invention, the at least one bioactive calcium phosphate phase comprises β-calcium pyrophosphate and α-tricalcium phosphate.

In one embodiment of the invention, the macroporous cement composition comprises 80-95 wt % hydroxyapatite, 0.1-10 wt % β-calcium pyrophosphate, preferably 1-10 wt % β-calcium pyrophosphate, and <10 wt % α-tricalcium phosphate.

In one embodiment of the invention, the step of obtaining the granules comprises:
mixing α-tricalcium phosphate, β-calcium pyrophosphate, and a sacrificial phase forming a dry powder mix, wherein all components are in solid form;
mixing the dry powder mix and a liquid forming a paste;
curing the paste at a temperature above room temperature, preferably at 50-60° C. for 20-30 hours;
terminating the chemical reaction associated with the curing after a predetermined time period selected so that a predetermined amount of α-tricalcium phosphate remains unreacted forming a solid cement composition, the predetermined amount being above 1 wt %, preferably above 3 wt %;
leaching the sacrificial phase from the solid cement composition; and
drying at a temperature above room temperature, preferably at 50-60° C. for 20-30 hours, to form the granules comprising the macroporous cement composition.

In one embodiment of the invention, the method further comprises a step of sterilizing the putty formulation.

In one embodiment of the invention, the step of sterilizing the putty formulation comprises sterilizing the putty formulation using irradiation.

Further aspects of the invention relates to a putty formulation according to the invention for use as a medicament, for use in treatment of a bone defect, for use as a bone void filler, or for use as a bone substitute.

The scope of the invention is defined by the claims. Any references in the description to methods of treatment refer to compounds, pharmaceutical compositions and medicaments of the present invention for use in a method for treatment of a human or animal body by therapy (or for diagnosis).

In the following, the invention will be described in more detail, by way of example only, with regard to non-limiting embodiments thereof, reference being made to the accompanying drawings.

### BRIEF DESCRIPTION OF THE DRAWINGS

FIG. 1 a) is a scanning electron microscope (SEM) image showing the macroporous cement composition in the form of granules according to the invention, b) is a graph showing the porosity at the y-axis against the amount of hydroxyapatite (HA) at the x-axis for four examples of the macroporous cement composition according to the invention;

FIG. 2 is a photograph showing a putty formulation according to one aspect of the invention;

FIG. 3 is a graph showing the amount of HA at the y-axis against the amount of coarse β-calcium pyrophosphate (β-CPP) at the x-axis for six examples of the macroporous cement composition according to the invention and two comparative examples;

FIG. 4 is a graph showing the pore size distribution for 10 different putty formulations as the pore size (μm) on the x-axis vs. the fraction or % of total porosity at the y-axis;

FIG. 5 a) and b) are SEM images showing the macroporous cement composition according to examples of the invention; and

FIG. 6 is a set of micro-computed tomography (p-CT) images showing the macroporous cement composition according to examples of the invention.

### DEFINITIONS AND ABBREVIATIONS

Bioactive—able to stimulate cells and/or form bonds between the tissue and the bioactive phase;
Biocompatible—able to be in contact with a living system, e.g., cells, or tissue, without producing adverse effects;
Macropores—pores with a diameter >100 μm;
kGy—abbreviation for kiloGrays
Vol %—abbreviation for volume precent, i.e., percentage of total volume;
Wt %—abbreviation for weight precent, i.e., percentage of total weight;
CMC—carboxymethyl cellulose
P407—poloxamer 407
HA—abbreviation for hydroxyapatite, chemical formula Ca₅(PO₄)₃(OH) or Ca₁₀(PO₃)₆(OH)₂;
β-CPP—abbreviation for β-calcium pyrophosphate, chemical formula Ca₂P₂O₇;
α-TCP—abbreviation for α-tricalcium phosphate, chemical formula Ca₃(PO₄)₂;
OCP—abbreviation for octacalcium phosphate, chemical formula Ca₈H₂(PO₄)₆·5H₂O; and
DCP—abbreviation for dicalcium phosphate, chemical formula Ca₂HPO₄ having 0 or 2H₂O.

### DETAILED DESCRIPTION

Adult humans have 206 different bones in their body. Bone is continuously remodeled during a person's lifetime, old and malfunctional bone is degraded and replaced with new bone. The cells present in bone are osteoclasts, osteoblasts, and osteocytes. They are responsible for the degradation and remodeling of bone. Ideally, a bone void filler material should function as a template for new bone formation rather than being a permanent bone substitute. Two main mechanisms are responsible for bone ingrowth into bone void fillers:
Osteoclastic degradation, i.e., that the bone void fillers are degraded in a similar way as natural bone and then replaced with new bone; and
Resorption through dissolution of the bone void filler material.

A faster bone ingrowth and/or a higher resorption rate can be achieved by the incorporation of macropores in the bone void filler material, i.e., pores with a diameter >100 μm. In addition, the composition of the bone void filler influences the bone ingrowth as well.

Typically, a bone void filler comprises one or more calcium phosphate phases. Examples of calcium phosphate phases include hydroxyapatite (HA, Ca₅(PO₄)₃(OH) or more commonly Ca₁₀(PO₃)₆(OH)₂), β-calcium pyrophosphate (β-CPP, Ca₂P₂O₇), α-tricalcium phosphate (α-TCP, Ca₃(PO₄)₂), octacalcium phosphate (OCP, Ca₈H₂(PO₄)₆·5H₂O), and dicalcium phosphate (DCP, Ca₂HPO₄ or Ca₂HPO₄·2H₂O). Different calcium phosphate phases have different properties, both in vitro and in vivo. Basically, all calcium phosphate phases are biocompatible and most of them are also bioactive. However, they have different dissolution/degradation rates in vivo (and in vitro). As mentioned above, this influences the rate of bone ingrowth and hence the effect of the bone void filler.

Hydroxyapatite (HA) is a form of calcium apatite with the formula Ca₅(PO₄)₃(OH) or Ca₁₀(PO₄)₆(OH)₂. Around 50 vol % of the human bone tissue is composed of hydroxyapatite. It is a widely studied material and suitable for use as a bone void filler or bone implant. Hydroxyapatite is known to be biocompatible and it is moderately bioactive. A bioactive calcium phosphate phase has the ability to stimulate cells and/or form bonds between the bone tissue and the bioactive phase, which is beneficial for a bone void filler. Hydroxyapatite does not degrade rapidly or release bioactive ions as rapidly as other, more soluble, calcium phosphates.

β-Calcium pyrophosphate is a bioactive phase, or bioactive material, that can react with the bone cells, or bone tissue. β-Calcium pyrophosphate is an insoluble calcium salt with the chemical formula Ca₂P₂O₇, it can be anhydrous or hydrous. Even if it is insoluble in vitro it is degraded quite rapidly in vivo where it can promote cell adhesion and tissue formation. It has been widely studied for use as a bone tissue repair material.

In order to be delivered into a bone void, the bone void filler material should be shaped into granules and mixed with a carrier in order to form a putty or paste that can be delivered into the bone void.

An aspect of the invention relates to a putty formulation comprising: granules comprising a macroporous cement composition comprising 80-95 wt % hydroxyapatite, Ca₁₀(PO₄)₆(OH)₂, wherein the balance comprises β-calcium pyrophosphate, Ca₂P₂O₇, and wherein the porosity of the composition is 60-80 vol % as determined using Archimedes method;
a liquid carrier, and
a binder material, wherein the average particle size of the granules is 50-800 μm as determined by sieving and/or scanning electron microscopy (SEM), and the portion of granules in the putty formulation is 25-50 wt %

The putty formulation of the invention is also referred to herein as a putty composition or simply a putty.

Hence, the macroporous cement composition comprises 80-95 wt % hydroxyapatite and β-calcium pyrophosphate.

A putty formulation according to the invention preferably comprises 20-50 wt % hydroxyapatite, more preferably 30-50 wt %, and most preferably 30-45 wt %, such as 35-45 wt % hydroxyapatite.

In one embodiment of the invention, the portion granules in the putty formulation is 30-50 wt %, more preferably 40-50 wt %. Hence, in such embodiment a putty formulation comprises 24-47.50 wt % hydroxyapatite.

In one embodiment of the invention, the portion of granules in the putty formulation is 25-45 wt %, preferably 30-40 wt %, and more preferably 30-35 wt %. Hence, in such embodiment a putty formulation comprises 24-38 wt % hydroxyapatite, more preferably 24-33.75 wt % hydroxyapatite.

The average particle size of the granules is 50-800 μm, preferably 53-800 μm, or more preferably 53-600 μm.

In one embodiment of the invention, the average particle size of the granules is determined by sieving.

In another embodiment of the invention, the average particle size of the granules is determined by SEM.

In a further embodiment of the invention, the average particle size of the granules is determined by sieving and SEM.

The average particle size of the granules could alternatively be determined by laser diffraction.

In an embodiment, about 5 g of granules have the following particle size distribution:
0.46 g of 50-100 μm;
0.91 g of 100-200 μm;
1.36 g of 200-400 μm;
1.14 g of 400-600 μm; and
1.14 g of 600-800 μm.

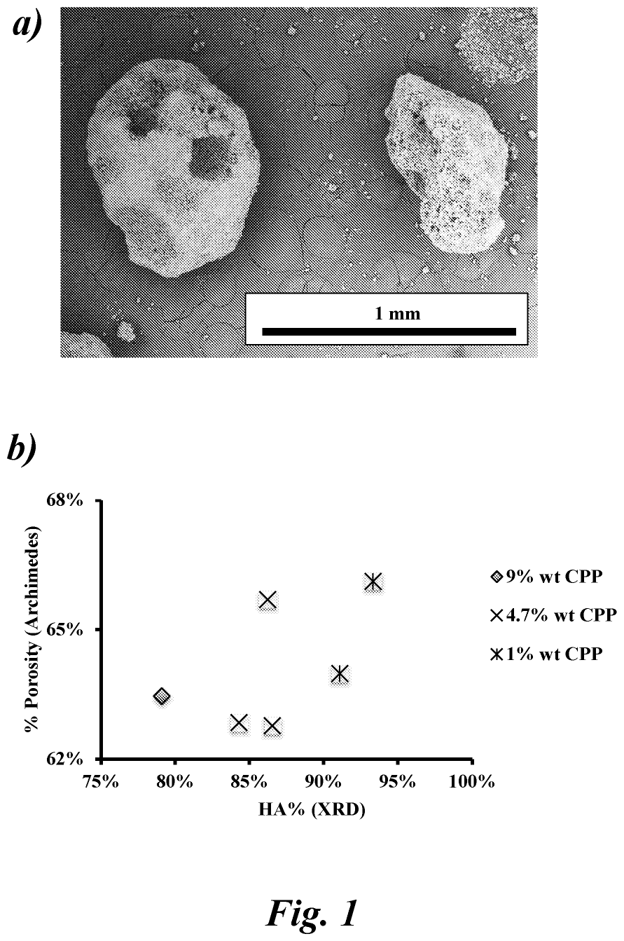

One example of granules according to the invention can be seen in the SEM image in FIG. 1a.

The term ‘porosity’ means total porosity, i.e., all volume in the macroporous cement composition that is empty space or voids, i.e., the total volume of pores that are equal to or below 800 μm, preferably equal to or below 600 μm in diameter. The porosity is given in vol % and calculated using helium (He) pycnometry or Archimedes method. Both He pycnometry and Archimedes method measures the skeletal or true density of a sample. True density is the ratio of the mass of solid material to volume of solid material (not accounting for closed pores). The true volume is measured by gas displacement using Boyle's law, for He pycnometry, or by liquid displacement (buoyancy) using Archimedes method. Helium, or another inert gas, is used as the displacement medium. The true density is calculated by dividing the sample weight by the true volume that is measured by He pycnometry, Archimedes method, or calculated from the densities of the component phases (Rietveld refinement). To determine the porosity the bulk or dry density, i.e., the theoretical density of the sample, calculated from the physical sample dimensions using device(s) like caliper(s), is divided with the true density calculated from the pycnometer or Archimedes measurement, see equation 1 below.

$$Porosity ( \% ) = 1 - ( {P}_{true} / {P}_{theoretical} ) \times 100 \qquad ( eq . 1 )$$

The aim of a bone void filler is to function as a template for new bone formation rather than being a permanent bone substitute. In order to do that it is an advantage if the cement composition, i.e., the bone void filler, comprises macropores. Macropores could improve cell colonization within the material and/or increase the osteoclastic degradation.

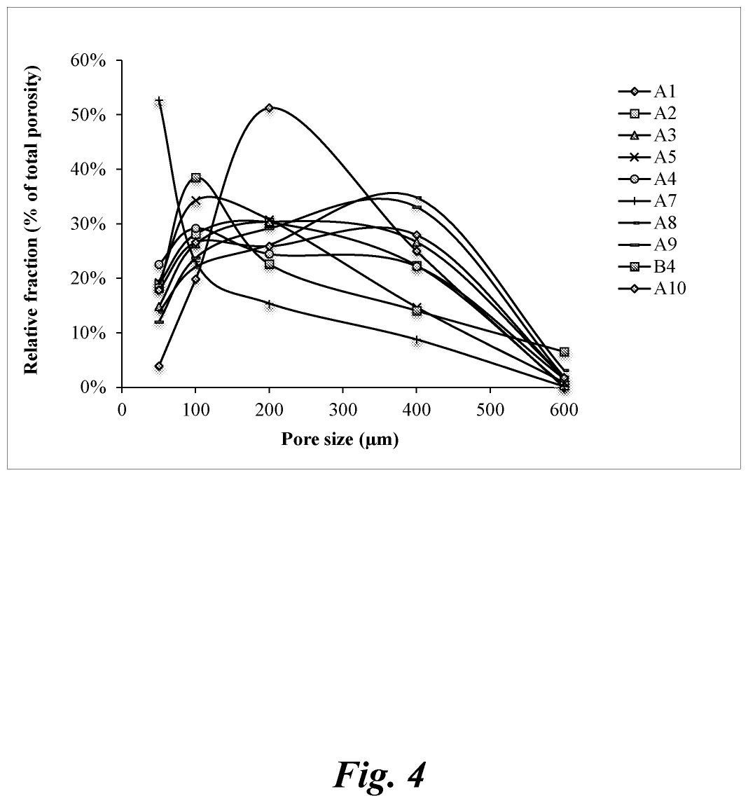

Macropores are defined as pores having a pore diameter >100 μm. In one embodiment of the invention, the macropores cement composition has pores with an average pore diameter >100 μm, preferably >150 μm, and more preferably >200 μm. The average pore diameter can be determined by, for example, micro-computed tomography (μ-CT), porosimetry, or any other suitable technique, which are known to the skilled person. Examples of pore size distributions for compositions according to the invention can be seen in FIG. 4. In FIG. 4, the pore size distribution for the different samples has been determined using micro-computed tomography (μ-CT) for pore size together with Archimedes method for total porosity. The porosity can be determined, for example by He pycnometry, as described above, or by micro-computed tomography (μ-CT), densitometry, or any other suitable technique as determined by the skilled person. In one embodiment of the invention, the porosity of the granules is 60-75 vol %, such as 60-70 vol %, preferably 62-68 vol %, or more preferably 63-67 vol %, as determined by Archimedes method. The graph in FIG. 1b shows the porosity values (in vol %) for four examples of macroporous cement composition according to the invention.

It is an advantage that the macroporous cement composition comprises a majority, e.g., 80 wt % or more, of hydroxyapatite, since it is a well-known phase that is stable, and its in vivo (e.g., rate of degradation and/or resorption) behavior has been studied and is reasonable well understood. It also has advantageous effects on the shelf-life and overall handling properties of the cement. It can be used as a delivery vehicle for other calcium phosphate phases that are beneficial to use in a bone cement.

Hydroxyapatite (HA) as used herein also include various forms of HA including, but not limited to, calcium deficient hydroxyapatite (CDHA), and mixtures of HA and CDHA.

One advantage with the present invention is that a putty formulation comprising hydroxyapatite and at least one additional calcium phosphate phase (bioactive calcium phosphate phase) is more bioactive than a single-phase cement with only hydroxyapatite. In one embodiment, the additional phase is β-calcium pyrophosphate (β-CPP). The additional phase may also be selected from β-calcium pyrophosphate, α-tricalcium phosphate, octacalcium phosphate, and dicalcium phosphate, including any combination thereof.

In one embodiment of the invention, the macroporous cement composition comprises hydroxyapatite, β-calcium pyrophosphate and α-tricalcium phosphate (α-TCP). α-TCP is the calcium salt of phosphoric acid, it has the chemical formula Ca₃(PO₄)₂ and it is a precursor of hydroxyapatite. It is a bioactive material that can be used as a bone replacement to enable the formation of new bones. α-TCP dissolves rapidly in vivo and releases ions, the rapid release is advantageous in terms of new bone formation.

In one embodiment of the invention, the macroporous cement composition comprises 80-95 wt % hydroxyapatite, Ca₁₀(PO₄)₆(OH)₂, 0.1-10 wt % β-calcium pyrophosphate, Ca₂P₂O₇, preferably 1.0-10 wt % β-calcium pyrophosphate, and <10 wt % α-tricalcium phosphate, Ca₃(PO₄)₂. Preferably the macroporous cement composition comprises around 90 wt % hydroxyapatite, Ca₁₀(PO₄)₆(OH)₂, 0.1-10 wt % β-calcium pyrophosphate, Ca₂P₂O₇, preferably 1.0-10 wt % β-calcium pyrophosphate, and <10 wt % α-tricalcium phosphate, Ca₃(PO₄)₂. As explained above, the granules of the putty formulation comprises the macroporous cement composition.

As understood by the skilled person it is possible that a macroporous cement composition additionally comprises minor amounts of impurities, such as salts, etc. The amount of impurities is typically <5 wt %, or <3 wt %, or <1 wt %.

There is a correlation between the behavior of a putty formulation, e.g., the flowability, adhesion, etc. and the granules to carrier ratio. The average size of the granules and the type of carrier additionally influences the behavior of the putty formulation.

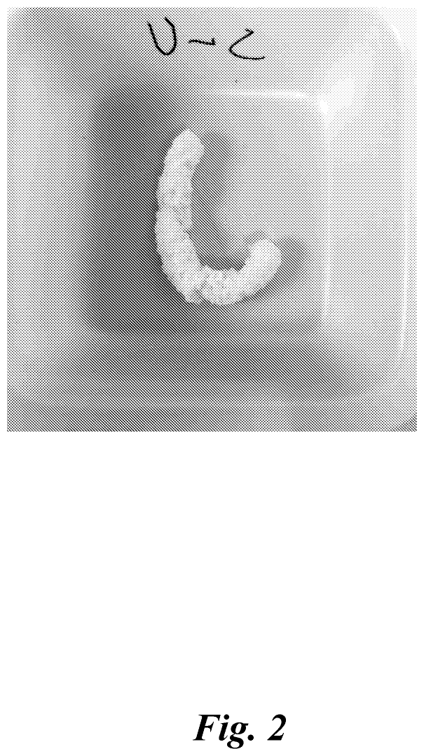

A putty formulation according to the invention remains substantially fixed and adhered in place without migrating into adjacent tissues upon placement. It is moldable, easy to handle and to place in the desired position. One example of such a putty formulation can be seen in FIG. 2.

The liquid carrier should be biocompatible, and not interact with the granules. In one embodiment, the liquid carrier is water. In one embodiment, the liquid carrier is an aqueous phosphate buffer, such as 5% Na₂HPO₄.

In one embodiment the putty formulation comprises 30-70 wt % liquid carrier, preferably 35-65 wt % liquid carrier.

In one embodiment the putty formulation comprises 50-70 wt % liquid carrier, or preferably 55-65 wt % liquid carrier.

In one embodiment, the putty formulation comprises 5-25 wt % of binder material, preferably 10-25 wt % binder material, more preferably 13-25 wt % binder material.

In one embodiment, the putty formulation comprises 5-10 wt % of binder material.

In one embodiment, the binder material comprises, or is, carboxymethyl cellulose (CMC), or poloxamer, or xantham gum, or chitosan. In a preferred embodiment, the binder material comprises, or is, CMC. In another preferred embodiment, the binder material comprises, or is, poloxamer, such as poloxamer 407.

Poloxamer is a triblock copolymer comprising, such as consisting of, a central hydrophobic block of polypropylene glycol (PPG) flanked by two hydrophilic blocks of polyethylene glycol (PEG). Poloxamer 407 comprises, on average, two PEG blocks of 101 repeat units and one PPG block of 56 repeat units. Poloxamer 407 is also known as PLURONIC®F-127, KOLLIPHOR® P 407 and SYNPERONIC® PE/F 127.

In vitro handling tests with different binder materials including CMC and poloxamers demonstrate comparable and desired handling characteristics. In vivo testing results demonstrate that poloxamers are comparatively more resistant to migration during and immediately after surgery. This is due to the thermoreversible properties of poloxamers when dissolved in water, i.e., fluid state at lower temperatures and gel state above sol-gel transition temperature at body temperature. This property causes a slight stiffening of the poloxamer-based putty formulation and its increased resistance to migration is a highly desired characteristic. Accordingly, preferred binder material is poloxamer, preferably poloxamer 407.

The liquid carrier and the binder material could be mixed together forming a gel. Hence, in an embodiment, the putty formulation comprises the granules and a gel comprised of the liquid carrier and the binder material.

In one embodiment, the putty formulation comprises 30-50 wt % granules, 5-20 wt % poloxamer 407, and 35-60 wt % water, preferably de-ionized or ultrapure water.

In one embodiment, the putty formulation comprises 30-35 wt % granules, 5-10 wt % CMC, and 40-60 wt % water, preferably de-ionized or ultrapure water.

In order to increase the shelf life of a putty formulation it could be advantageous to include an antioxidant to prevent oxidation of the putty formulation. In one embodiment, the putty formulation further comprises an antioxidant. The antioxidant may, for example, be vitamin C or ascorbic acid. In one embodiment, the putty formulation comprises 0.2-2.0 wt %, or preferably 0.5-1.5 wt %, antioxidant.

A putty formulation according to the invention may be used in treatments of bone fracture, or for bone fracture healing, or joint fusion, bone voids, etc. Prior to such a use, the putty formulation should be sterilized. A putty formulation is preferably sterilized once it is contained within a syringe or similar, i.e., in the final form, in which it is delivered to the medical doctor, veterinarian, surgeon, etc. In one embodiment, the putty formulation is sterilized by means of irradiation, preferably gamma irradiation. In one embodiment, the putty formulation is sterilized by using 25-36 kGy gamma irradiation.

Another aspect of the invention relates to a method for manufacturing a putty formulation. The method comprises the steps of:
obtaining granules, wherein the granules comprises:
   80-95 wt % hydroxyapatite (Ca₁₀(PO₄)₆(OH)₂), wherein the balance comprises at least one bioactive calcium phosphate phase, and wherein the porosity of the composition is 60-80 vol % as determined by Archimedes method;
   wherein the granules have an average particle size of 50-800 μm, as determined by sieving and/or SEM; and
mixing the granules with a liquid carrier, for example water or 5% Na₂HPO₄, and a binder material, wherein the portion of granules in the formulation is 25-50 wt %.

In one embodiment, the mixing step comprises mixing the granules with a gel comprised of or formed by the liquid carrier and the binder material.

In one embodiment, the at least one bioactive calcium phosphate phase comprises β-calcium pyrophosphate, preferably comprises β-calcium pyrophosphate and a-tricalcium phosphate.

In one embodiment, the macroporous cement composition comprises:
80-95 wt % hydroxyapatite;
0.1-10 wt % β-calcium pyrophosphate, preferably 1.0-10 wt % β-calcium pyrophosphate; and
<10 wt % α-tricalcium phosphate.

In one embodiment, the granules have a total porosity of 60-75 vol % as determined by Archimedes method.

In one embodiment, the step of obtaining granules comprises the steps of:
First mixing step: mixing of α-tricalciumphosphate (α-TCP), β-calcium pyrophosphate (β-CPP), hydroxyapatite (HA) and a sacrificial phase forming a dry powder mix, wherein all components are in a solid form;
Second mixing step: mixing the dry powder mix formed in the first mixing step and a liquid and forming a paste;
Curing step: curing the paste at a temperature above room temperature, preferably at 50-60° C. for 20-30 hours;
Termination step: terminating, i.e., halting of, the chemical reaction associated with the curing after a predetermined time period selected so that a predetermined amount of α-tricalcium phosphate (α-TCP) remains unreacted forming a solid cement composition, wherein the predetermined amount being above 1 wt %, preferably above 3 wt %;
Leaching step: leaching the sacrificial phase solid cement composition; and
Drying step: drying at a temperature above room temperature, preferably at 50-60° C. for 20-30 hours, to form the granules comprising the macroporous cement composition.

The curing step may be performed at 50-60° C. for 20-30 hours, and the drying step may be performed at 50-60° C. for 20-30 hours. The termination step may be performed by submerging the first cement formed in the curing step in a solvent. Hence, in an embodiment, terminating the chemical reaction associated with the curing comprises submerging a cement formed by curing the paste in a solvent, such as acetone, isopropanol and/or ethanol, or by freezing the composition to between −20 to −80° C. for around 4 hours or longer.

The sacrificial phase may be polyethylene glycol (PEG), the liquid may be water, and the amount of water in the second mixing step may be 0.4-0.6 mL/g of the dry powder mix.

In one embodiment, the average particle size of the (β-calcium pyrophosphate particles is 500 nm-10 μm as determined by sieving and/or SEM.

In one embodiment, the binder material is CMC, and the amount of binder material is 5-10 wt %. In another embodiment, the binder material is poloxamer 407, and the amount of binder material is 5-25 wt %, preferably 10-25 wt %, and more preferably 13-25 wt %. In one embodiment, the liquid carrier is water, and the amount of liquid carrier is 30-70 wt %, such as 50-70 wt %, preferably 35-65 wt %, such as 55-65 wt %.

As explained above, the putty formulation is preferably sterilized prior to use. In one embodiment of the method of manufacture, the method comprises an additional step of sterilization after the mixing step. Optionally, the putty formulation may be placed in a container, e.g., a syringe, prior to the sterilization step. The sterilization may be performed using gamma irradiation or any other suitable technique known to the skilled person.

As an example, during use of a putty formulation according to the invention, a syringe comprising a sterilized formulation is delivered to a surgeon. The surgeon delivers the putty formulation into a bone void by using the syringe. Once in place in the bone void, the putty formulation stays in position. By the bone generation processes the putty formulation is degraded and replaced with new bone. A putty formulation according to the invention is suitable for treatments of bone defects, such as for example fractures or osteotomy, both in humans and in animals (vertebrates).

The present invention also relates to a putty formulation according to the invention for use as a medicament, and in particular for use in treatment of a bone defect, for use as a bone void filler and/or for use as a bone substitute.

The invention also provides the use of a putty formulation according to the invention for the manufacture of a medicament for treatment of a bone defect.

The invention further relates to a method for treating a bone defect. The method comprises delivering a putty formulation according to the invention into a bone void in a subject, preferably using a syringe.

As used herein, the terms “treat”, “treating” and “treatment” are taken to include an intervention performed with the intention of preventing the development or altering the pathology of a disorder or symptom. Accordingly, “treatment” refers to both therapeutic treatment and prophylactic or preventative measures, wherein the object is to prevent or slow down (lessen) the targeted disorder or symptom. Accordingly, the term “treating” encompasses treating and/or preventing the development of a disorder or symptom. The invention may therefore be useful for preventing a bone defect in a subject, such as by preventing a worsening of the symptoms of a bone defect.

As used here in the term “subject” refers to an individual, e.g., a human or an animal (vertebrate), preferably a mammal.

### EXAMPLES

### Example 1

27 samples were prepared for the study. Of these 24 were sterilized by Gamma irradiation. There were four formulation variants: U-A, U-B, U-C, and U-D, see Table 1.

Three samples from each variant were used in mechanical testing, three samples from each variant were used for accelerated testing, and the three non-sterilized samples were used as controls (Ctrl).

TABLE 1
Compositions of test putty formulations

| Sample | Particles | | | |
|---|---|---|---|---|
| ID | (50-600 μm) | CMC | H₂O | Vit C* |
| U-A | 1.666 g | 0.192 g | 2.5 mL | 0 g |
| U-B | 1.666 g | 0.192 g | 2.5 mL | 0.066 g |
| U-C | 1.666 g | 0.383 g | 3.06 mL | 0 g |
| U-D | 1.666 g | 0.383 g | 3.33 mL | 0.069 g |
| Ctrl | 1.666 g | 0.192 g | 2.5 mL | 0 g |

* L-ascorbic acid

### 1.1: Irradiation Test

### Test 1: Glove Testing

### Criteria for Passing the Test:

putty formulation forms a cohesive ball
putty formulation forms a cohesive sausage
ball remain cohesive and is easily molded
particles remain within putty formulation with minimal sticking to gloves upon manipulation

TABLE 2
Results of glove testing

| | Total | | | | | | |
|---|---|---|---|---|---|---|---|
| | granule | D.I. | Irradiation | | | | |
| Sample | mass | water | dosage | CMC | Vit C | | |
| no | (g) | (mL) | (kGy) | (g) | (g) | Remarks | Pass/fail |
| U-A1 | 1.666 | 2.5 | 25-36 | 0.192 | 0 | Too soft to work into putty | FAIL |
| U-A2 | 1.666 | 2.5 | 25-36 | 0.192 | 0 | Not cohesive | FAIL |
| U-A3 | 1.666 | 2.5 | 25-36 | 0.192 | 0 | Easy to remove from vial | FAIL |
| U-B2 | 1.666 | 2.5 | 25-36 | 0.192 | 0.066 | Minimal glove residual | PASS |
| U-B3 | 1.666 | 2.5 | 25-36 | 0.192 | 0.066 | Good result, cohesive | PASS |
| U-C1 | 1.666 | 3.06 | 25-36 | 0.383 | 0 | Workable into cohesive | PASS |
| | | | | | | sausage shape | |
| U-C2 | 1.666 | 3.06 | 25-36 | 0.383 | 0 | Workable, large residue | PASS |
| U-C3 | 1.666 | 3.06 | 25-36 | 0.383 | 0 | Nice handling feel, not too | PASS |
| | | | | | | rubbery | |
| U-D1 | 1.666 | 3.33 | 25-36 | 0.383 | 0.069 | A bit tacky in gloves, | PASS |
| | | | | | | workable | |
| U-D2 | 1.666 | 3.33 | 25-36 | 0.383 | 0.069 | Large particle residue | PASS |
| U-Ctrl1 | 1.666 | 2.5 | 0 | 0.192 | 0 | Binds very well | PASS |
| U-Ctrl2 | 1.666 | 2.5 | 0 | 0.192 | 0 | Zero residue on gloves | PASS |
| U-Ctrl3 | 1.666 | 2.5 | 0 | 0.192 | 0 | Possible dried in vial | PASS |

All U-A samples failed the glove test. All U-B, U-C and U-D passed the glove test. All the control samples passed the test.

### Test 2: Injectability Testing

TABLE 3
Results of injectability testing

| | Total | | | | | | | |
|---|---|---|---|---|---|---|---|---|
| | granule | D.I. | Irradiation | | | | | |
| Sample | mass | water | dosage | CMC | Vit C | Fₘₐₓ | Displacement | |
| no | (g) | (mL) | (kGy) | (g) | (g) | (N) | (mm) | Remarks |
| U-A1 | 1.666 | 2.5 | 25-36 | 0.192 | 0 | 57.9 | 25 | Very little to inject |
| U-A2 | 1.666 | 2.5 | 25-36 | 0.192 | 0 | 38.5 | 25 | Greater consistency |
| U-A3 | 1.666 | 2.5 | 25-36 | 0.192 | 0 | 34.2 | 25 | Most cohesive |
| U-B1 | 1.666 | 2.5 | 25-36 | 0.192 | 0.066 | 36.5 | 25 | Complete liquid |
| U-B2 | 1.666 | 2.5 | 25-36 | 0.192 | 0.066 | 21.1 | 25 | Much more solid |
| | | | | | | | | injection |
| U-B3 | 1.666 | 2.5 | 25-36 | 0.192 | 0.066 | 38.8 | 25 | Consistent flow |
| U-C1 | 1.666 | 3.06 | 25-36 | 0.383 | 0 | 32.1 | 25 | Very good flow, good |
| | | | | | | | | consistency |
| U-C2 | 1.666 | 3.06 | 25-36 | 0.383 | 0 | 51.2 | 25 | Large force required |
| | | | | | | | | for injection |
| U-C3 | 1.666 | 3.06 | 25-36 | 0.383 | 0 | 45.6 | 25 | Cracking during |
| | | | | | | | | injection |
| U-D1 | 1.666 | 3.33 | 25-36 | 0.383 | 0.069 | 12.3 | 25 | Smooth flow from |
| | | | | | | | | syringe |
| U-D2 | 1.666 | 3.33 | 25-36 | 0.383 | 0.069 | 11.8 | 25 | Very sticky and tacky |
| U-D3 | 1.666 | 3.33 | 25-36 | 0.383 | 0.069 | 15.2 | 25 | No remark |
| U-Ctrl1 | 1.666 | 2.5 | 0 | 0.192 | 0 | 31.3 | 25 | Much easier to claim |
| | | | | | | | | from syringe |
| U-Ctrl2 | 1.666 | 2.5 | 0 | 0.192 | 0 | 35.5 | 25 | No tackiness with |
| | | | | | | | | putty |
| U-Ctrl3 | 1.666 | 2.5 | 0 | 0.192 | 0 | 37.4 | 25 | No remark |

The U-A samples were dripping from the syringe prior to applying any force. The formulations were easily injectable but hard to control.

The U-B1 sample was similar to the U-A samples, very watery. U-B2 and U-B3 had a good cohesive flow out of the syringe, with a controllable flow. It was clear that Vitamin C had a positive effect of reducing the degradation of CMC.

The U-C samples all had very consistent, cohesive flow. All were easy to work with and injectable.

The U-D samples all had very consistent, cohesive flow. All were easy to work with and injectable.

All control samples were easy to work with, having consistent, cohesive flow. They were most similar to the U-C samples.

### Test 3: Mechanical Texture Testing

TABLE 4
Results of mechanical texture testing

| | Total | D.I. | Irradiation | | | | |
|---|---|---|---|---|---|---|---|
| Sample | granule | water | dosage | CMC | Vit C | Fₘₐₓ | |
| no | mass (g) | (mL) | (kGy) | (g) | (g) | (N) | Remarks |
| U-A1 | 1.666 | 2.5 | 25-36 | 0.192 | 0 | N/A | Cannot form cube shape, |
| | | | | | | | too much material lost. |
| U-A2 | 1.666 | 2.5 | 25-36 | 0.192 | 0 | N/A | N/A |
| U-A3 | 1.666 | 2.5 | 25-36 | 0.192 | 0 | N/A | N/A |
| U-B1 | 1.666 | 2.5 | 25-36 | 0.192 | 0.066 | N/A | N/A |
| U-B2 | 1.666 | 2.5 | 25-36 | 0.192 | 0.066 | 0.842 | Forms a solid shape |
| U-B3 | 1.666 | 2.5 | 25-36 | 0.192 | 0.066 | 1.11 | Holds good deformation |
| U-C1 | 1.666 | 3.06 | 25-36 | 0.383 | 0 | 3.69 | Strong mechanical texture |
| | | | | | | | strength |
| U-C2 | 1.666 | 3.06 | 25-36 | 0.383 | 0 | 2.83 | Holds good shape |
| U-C3 | 1.666 | 3.06 | 25-36 | 0.383 | 0 | 3.40 | Minimal tackiness |
| U-D1 | 1.666 | 3.33 | 25-36 | 0.383 | 0.069 | 1.30 | Very easy to mould, low |
| | | | | | | | mechanical strength |
| U-D2 | 1.666 | 3.33 | 25-36 | 0.383 | 0.069 | 1.69 | Very sticky to glove |
| U-D3 | 1.666 | 3.33 | 25-36 | 0.383 | 0.069 | 0.792 | Low strength |
| U-Ctrl1 | 1.666 | 2.5 | 0 | 0.192 | 0 | 7.08 | No remark |

during

| | | | | | | | |
|---|---|---|---|---|---|---|---|
| U-Ctrl2 | 1.666 | 2.5 | 0 | 0.192 | 0 | 8.18 | Cracking during |
| | | | | | | | compression |
| U-Ctrl3 | 1.666 | 2.5 | 0 | 0.192 | 0 | 8.77 | High strength |

The U-A formulations and the U-B1 formulation could not be tested, the putties could not be formed into shapes.

The U-B2 and U-B3 samples were easy formed into shapes. They had good deformation and ability to hold its deformed shape post compression. The samples had poor mechanical strength.

The U-C samples were moderately easy to form into shape. They had good deformation abilities and were able to hold their deformed shaped post compression.

The U-D samples were easy to form into shapes. They had good deformation but were unable to hold their deformed shape post compression.

The control samples very easy to form into shapes and had good deformation properties. They were able to hold their shapes post compression. The control samples were most similar to the U-C samples.

TABLE 5
Summary of all results in irradiation test

| | Glove | Injectability | Mechanical texture |
|---|---|---|---|
| Test | test | test | test |
| Formula A | Failed to bind | Injected as a very | Had very little |
| U-A | together. Not | liquid material. | mechanical stability. |
| | workable | | |
| Formula B | Not cohesive. | Inconsistent results; | Inconsistent results; |
| U-B | Inconsistent | liquid and putty | low mechanical |
| | results. | texture. | stability. |
| Formula C | Binds very well. | Injected very well | Had a firm mechanical |
| U-C | Small residue left | as a solid putty, | stability. |
| | on gloves. | controlled flow. | |
| Formula D | Vary easy to | Injected very well | Had a low/moderate |
| U-D | shape. Small | as a solid putty, | mechanical stability. |
| | residue left on | controlled flow. | |
| | gloves. | | |
| Control | Very easy to | Injected very well | Had a firm |
| U-Ctrl | shape. Zero/ | as a solid putty, | mechanical stability. |
| | small residue. | controlled flow. | |

From the test it appears that Formula C followed by Formula D had the best performance in terms of handling and strength. They were also most similar to the control samples.

### 1.2: Accelerated Ageing Test

For the ageing test the samples were incubated at 45° C. for 67 days, representing 12 month accelerated ageing.

### Test 1: Glove Testing

### Criteria for Passing the Test:

putty formulation forms a cohesive ball
putty formulation forms a cohesive sausage
ball remain cohesive and is easily molded
particles remain with putty formulation with minimal sticking to gloves upon manipulation

TABLE 6
Results of glove testing

| | Total | | | | | | |
|---|---|---|---|---|---|---|---|
| | granule | D.I. | Irradiation | | | | |
| Sample | mass | water | dosage | CMC | Vit C | | |
| no | (g) | (mL) | (kGy) | (g) | (g) | Remarks | Pass/fail |
| U-A5 | 1.666 | 2.5 | 25-36 | 0.192 | 0 | Completely turned to dust | FAIL |
| U-A6 | 1.666 | 2.5 | 25-36 | 0.192 | 0 | Improvement over U-A5, | FAIL |
| | | | | | | however still very dry | |
| U-A7 | 1.666 | 2.5 | 25-36 | 0.192 | 0 | Able to form sausage shape | PASS |
| U-B5 | 1.666 | 2.5 | 25-36 | 0.192 | 0.066 | Crumbled while making | FAIL |
| | | | | | | sausage shape, large amount | |
| | | | | | | of cracking. | |
| U-B6 | 1.666 | 2.5 | 25-36 | 0.192 | 0.066 | Minimal cracking, however | PASS |
| | | | | | | glove residue. | |
| U-B7 | 1.666 | 2.5 | 25-36 | 0.192 | 0.066 | Minimal cracking and | PASS |
| | | | | | | minimal residue. | |
| U-C5 | 1.666 | 3.06 | 25-36 | 0.383 | 0 | No residue, very easy to form. | PASS |
| | | | | | | No cracks. | |
| U-C6 | 1.666 | 3.06 | 25-36 | 0.383 | 0 | No cracks, minimal residue. | PASS |
| U-C7 | 1.666 | 3.06 | 25-36 | 0.383 | 0 | No cracks, chalking residue | PASS |
| U-D5 | 1.666 | 3.33 | 25-36 | 0.383 | 0.069 | Minimal residue and no | PASS |
| | | | | | | cracks. Elastic feel. | |
| U-D6 | 1.666 | 3.33 | 25-36 | 0.383 | 0.069 | Large amount of residue on | PASS |
| | | | | | | gloves | |
| U-D7 | 1.666 | 3.33 | 25-36 | 0.383 | 0.069 | No cracks, minimal residue. | FAIL |

The U-A5 sample was completely dry and hence failed the glove test. U-A6 was able to bind into a ball but too dry to form into a sausage shape, however it also failed the test. The U-A7 sample passed the test, it was able to form into a sausage shape. In total the U-A samples failed the accelerated ageing test.

The U-B samples were formable but started to form cracks when rolled into sausage shapes. The samples passed the accelerated ageing test.

The U-C samples bonded very well together and were easy to form without any crack formation. The samples passed the accelerated ageing test.

The U-D samples bonded very well together and were easy to form without any crack formation. The samples passed the accelerated ageing test.

### Test 2: Injectability Testing

TABLE 7
Results of injectability testing

| | Total | | | | | | | |
|---|---|---|---|---|---|---|---|---|
| | granule | D.I. | Irradiation | | | | | |
| Sample | mass | water | dosage | CMC | Vit C | Fₘₐₓ | Displacement | |
| no | (g) | (mL) | (kGy) | (g) | (g) | (N) | (mm) | Remarks |
| U-A1 | 1.666 | 2.5 | 25-36 | 0.192 | 0 | 11.5 | 23 | Complete crumble, dust |
| U-A2 | 1.666 | 2.5 | 25-36 | 0.192 | 0 | 28.9 | 23 | Injecting completely out |
| | | | | | | | | and as a sausage shape |
| U-A3 | 1.666 | 2.5 | 25-36 | 0.192 | 0 | 39.8 | 23 | Poor injection, wet |
| | | | | | | | | chunks |
| U-B1 | 1.666 | 2.5 | 25-36 | 0.192 | 0.066 | 103 | 23 | Dry putty |
| U-B2 | 1.666 | 2.5 | 25-36 | 0.192 | 0.066 | 127 | 23 | Very high f-max |
| U-B3 | 1.666 | 2.5 | 25-36 | 0.192 | 0.066 | 63.4 | 23 | Consistent flow |
| U-C1 | 1.666 | 3.06 | 25-36 | 0.383 | 0 | 23.3 | 23 | Smooth flow |
| U-C2 | 1.666 | 3.06 | 25-36 | 0.383 | 0 | 54.3 | 23 | High f-max |
| U-C3 | 1.666 | 3.06 | 25-36 | 0.383 | 0 | 34.4 | 23 | Smooth sausage shape |
| U-D1 | 1.666 | 3.33 | 25-36 | 0.383 | 0.069 | 31.9 | 23 | Slight cracking present |
| U-D2 | 1.666 | 3.33 | 25-36 | 0.383 | 0.069 | 24.3 | 23 | Samples are very dark in |
| | | | | | | | | color |
| U-D3 | 1.666 | 3.33 | 25-36 | 0.383 | 0.069 | 45.9 | 23 | No remark |

The U-A5 sample were completely dry and could not be injected. The UA-6 sample injected very well. The UA-7 sample injected poorer and came out less homogenous than the U-A6 sample.

The U-B5 sample were slightly dry and cracked during injection. U-B6 and U-B7 injected well.

All U-C samples had very consistent and cohesive flows. All were injectable with little waste in the syringe.

All U-D samples were similar to the U-C samples.

### Test 3: Mechanical Texture Testing

TABLE 8
Results of mechanical texture testing

| | Total | D.I. | Irradiation | | | | |
|---|---|---|---|---|---|---|---|
| Sample | granule | water | dosage | CMC | Vit C | Fₘₐₓ | |
| no | mass (g) | (mL) | (kGy) | (g) | (g) | (N) | Remarks |
| U-A1 | 1.666 | 2.5 | 25-36 | 0.192 | 0 | N/A | Too crumbly to perform the |
| | | | | | | | test |
| U-A2 | 1.666 | 2.5 | 25-36 | 0.192 | 0 | 8.00 | Very easy to form into shape |
| U-A3 | 1.666 | 2.5 | 25-36 | 0.192 | 0 | 14.5 | High F-max compressive |
| | | | | | | | force |
| U-B1 | 1.666 | 2.5 | 25-36 | 0.192 | 0.066 | 9.58 | Sticking to probe, slightly |
| | | | | | | | cracking |
| U-B2 | 1.666 | 2.5 | 25-36 | 0.192 | 0.066 | 10.3 | Sticking to probe |
| U-B3 | 1.666 | 2.5 | 25-36 | 0.192 | 0.066 | 5.40 | Sticking to probe |
| U-C1 | 1.666 | 3.06 | 25-36 | 0.383 | 0 | 7.91 | Sticking to probe |
| U-C2 | 1.666 | 3.06 | 25-36 | 0.383 | 0 | 11.3 | Sticking to probe |
| U-C3 | 1.666 | 3.06 | 25-36 | 0.383 | 0 | 5.32 | Good retention of |
| | | | | | | | deformation |
| U-D1 | 1.666 | 3.33 | 25-36 | 0.383 | 0.069 | 4.05 | Low f-max, weak |
| | | | | | | | mechanical strength. |
| | | | | | | | Slightly tacky to touch. |
| U-D2 | 1.666 | 3.33 | 25-36 | 0.383 | 0.069 | 3.13 | Retains shape of |
| | | | | | | | compression well. |
| U-D3 | 1.666 | 3.33 | 25-36 | 0.383 | 0.069 | 3.56 | Sticking to probe |

The U-A5 sample could not conduct the test due to the putty being too dry and crumbly to form into shape. The U-A6 and U-A7 samples were very moldable and demonstrated high strength.

The U-B samples demonstrated overall good deformation. They adhered to the compression probe after testing, however the samples were not tacky or sticky to handle.

The U-C samples very easy to form. They adhered to the compression probe after testing, however the samples were not tacky or sticky to handle. The samples showed no signs of cracking.

The U-D samples were similar to the U-C samples. They were easy to shape and not too sticky or tacky too handle.

TABLE 9
Summary of all results in accelerated ageing test

| | | | Mechanical texture |
|---|---|---|---|
| Test | Glove test | Injectability test | test |
| Formula A | Inconsistent results, | Inconsistent results | Inconsistent results, |
| U-A | moderate glove | | however |
| | residue | | demonstrated high |
| | | | mechanical strength |
| Formula B | Cohesive putty, | Good, cohesive flow, | Firm mechanical |
| U-B | moderate cracking, | consistent results | stability, good |
| | minimal residue | | deformation |
| Formula C | Binds very well, | Injected very well as a | Firm mechanical |
| U-C | minimal/zero | solid putty, controlled | stability, good |
| | residue on gloves | flow | deformation |
| Formula D | Binds very well, | Injected very well, | Moderate/weak |
| U-D | moderate residue | slight cracking on | mechanical |
| | on gloves | injected putty | stability. |

The results from the accelerated ageing test indicate that there is no great change to the putty over a 12-month shelf life.

In summary, the U-A, U-B and U-C samples mirrored the unaged and unsterilized control samples best.

### Example 2

A set of samples were prepared according to the method described above. In brief, in the first step, 1000 mg α-TCP (d₅₀≤6.12 μm, obtained from Innotere) was mixed with 250-1000 mg PEG (100-600 μm), 10 mg HA seed crystals (particle size <0.063 μm) and 10-140 mg β-CPP. Two types of β-CPP were used, one coarse (d₅₀=8.24 μm) and one fine (d₅₀=1.55 μm). In the second step, the powder obtained in the first step was mixed with deionized water using a L/P of 0.4-0.6. The composition was cured at 50-60° C. for 20-30 hours, after which it was submerged in ethanol in the termination step. The PEG (the sacrificial phase) was removed by submerging the composition in water (70-90° C.) for ˜24 hours. Finally, the composition was dried at 40° C. for 24 hours.

A summary of the formed samples is shown in Table 10 below.

TABLE 10
Summary of prepared samples

| | | PEG | | | | | |
|---|---|---|---|---|---|---|---|
| | | (1-600 | β-CPP | β-CPP | | | |
| Sample | α-TCP | μm) | (coarse) | (fine) | HA | | L/P |
| name | [mg] | [mg] | [mg] | [mg] | [mg] | Liquid | [μL] |
| A1 | 1000 | 250 | 140 | 10 | 10 | Water | 600 |
| A2 | 1000 | 250 | 90 | 10 | 10 | Water | 600 |
| A3 | 1000 | 250 | 40 | 10 | 10 | Water | 600 |
| A4 | 1000 | 250 | 0 | 10 | 10 | Water | 600 |
| A5 | 1000 | 250 | 0 | 50 | 10 | Water | 600 |
| A6 | 1000 | 250 | 0 | 100 | 10 | Water | 600 |
| A7 | 1000 | 250 | 50 | 0 | 10 | Water | 600 |
| A8 | 1000 | 250 | 100 | 0 | 10 | Water | 600 |
| A9 | 1000 | 250 | 10 | 10 | 10 | Water | 600 |
| A10 | 1000 | 250 | 25 | 25 | 10 | Water | 600 |
| A11 | 1000 | 250 | 50 | 50 | 10 | Water | 600 |
| B1 | 1000 | 1000 | 0 | 10 | 10 | Water | 400 |
| B2 | 1000 | 1000 | 0 | 10 | 10 | Water | 600 |
| B3 | 1000 | 0 | 0 | 10 | 10 | Water | 400 |
| B4 | 1000 | 0 | 0 | 10 | 10 | Water | 600 |
| B5 | 1000 | 250 | 0 | 0 | 10 | Water | 600 |

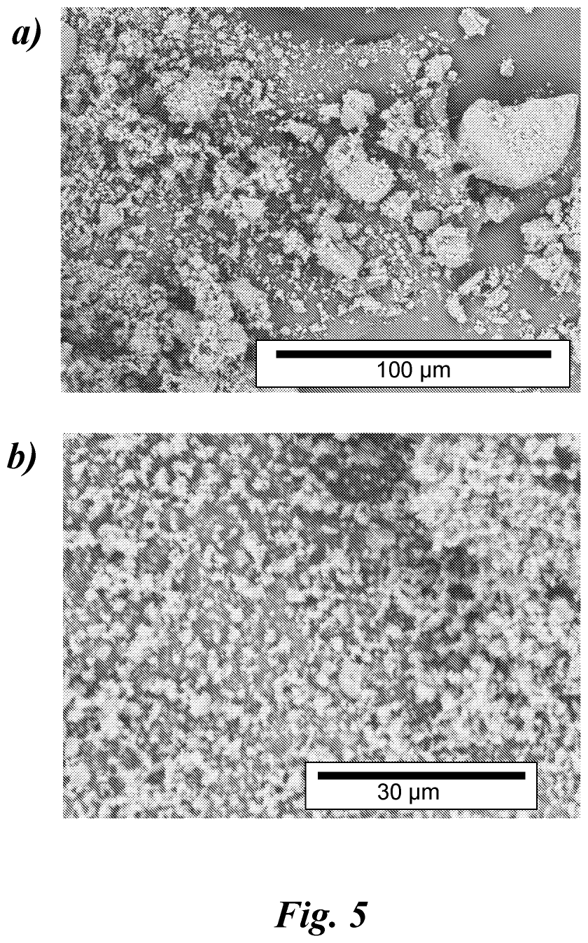

Sample A6 and A11 comprising 100% and 50% fine p-CPP, respectively did not set and were, hence, not analyzed further. The graph in FIG. 4 shows this, that when the amount of coarse β-CPP is too little almost no HA is formed, i.e., the sample does not set. Hence, large amounts of fine β-CPP inhibits the HA reaction. SEM images of coarse respectively fine β-CPP particles can be seen in FIG. 5, FIG. 5a shows an image of coarse particles and FIG. 5b shows an image of fine particles.

The B-samples (B1-B5) all comprise a larger amount of PEG and a lesser amount of β-CPP. Samples containing more than 40% PEG did not set, consistently. The A1, A6 and B2 samples did not set and were, hence, not analyzed further.

The remaining samples were analyzed for porosity and composition. The composition was analyzed using XRD and Rietveld refinement. The porosity was analyzed using Archimedes principle wherein wet density is compared to dry density, He pycnometry, XRD and μCT.

The results from the analyzed samples are summarized in Table 11 below, and in FIG. 1b that is a graph showing amount of HA vs Archimedes porosity.

TABLE 11
Summary of results from composition and porosity analysis.

| | | HA | Archimedes | Pycnometer | XRD |
|---|---|---|---|---|---|
| Sample | B-CPP | (formed) | porosity | porosity | porosity |
| name | [wt %] | [wt %] | [vo1 %] | [vol %] | [vo1 %] |
| A2 | 9.0 | 83.0 | 62.3 | — | — |
| A3 | 4.7 | 86.9 | 63.3 | — | — |
| A4 | 1.0 | 93.3 | 66.1 | 68.7 | 72.0 |
| A5 | 4.7 | 84.3 | 62.8 | 67.2 | 70.3 |
| A7 | 4.7 | 86.2 | 65.7 | 67.8 | 70.9 |
| A8 | 9.0 | 79.1 | 63.5 | 66.6 | 69.7 |
| A9 | 1.0 | 91.1 | 64.0 | 68.4 | 71.6 |
| A10 | 4.7 | 86.5 | 62.8 | 67 | 70.2 |
| B1 | | | | | |
| B3 | | | | | |
| B4 | 1.0 | 94 | 58.8 | — | — |
| B5 | | | | | |

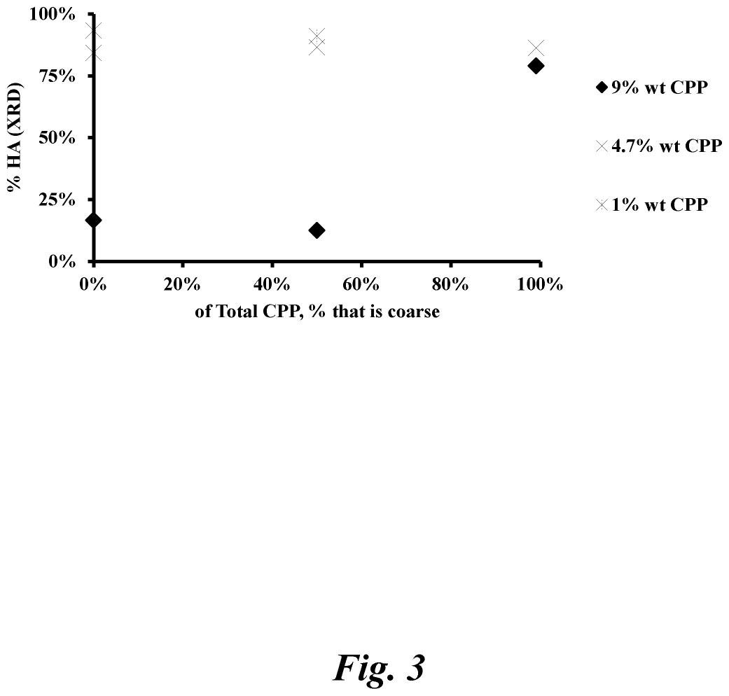

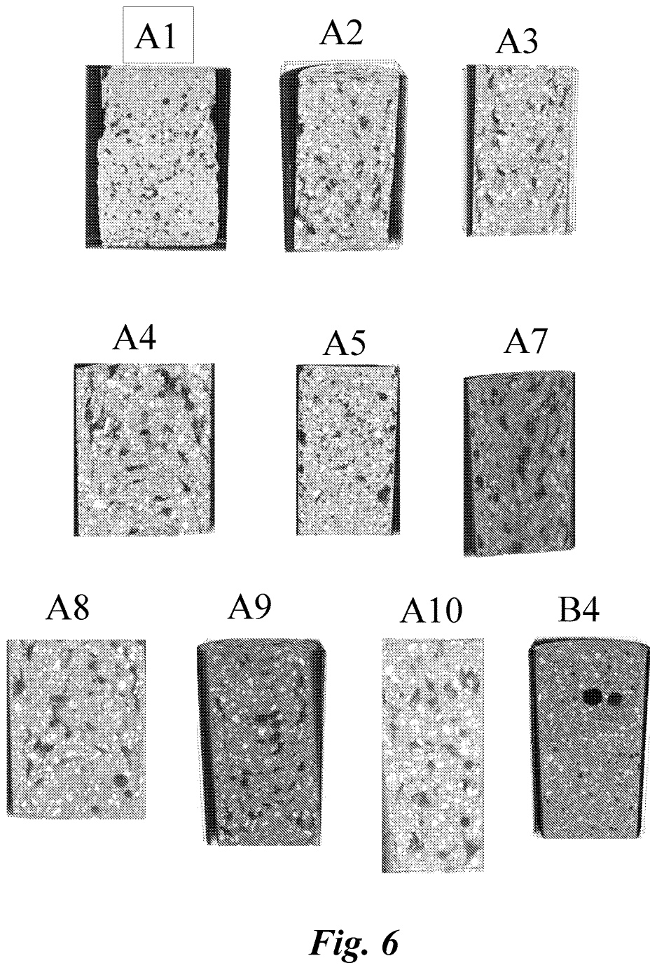

The samples were further analyzed using a μCT in order to visualize the porous structure and determine the pore size distribution (same method as in WO 2015/162597). The results can be seen in FIGS. 3 and 6. FIG. 6 shows the images, as can be seen in the figure all analyzed samples have visible pores. FIG. 7 shows the pore size distributions for the different samples, as can be seen all samples has pores with pore diameters between 50-600 m and an average pore size around 250 m.

### Example 3

A putty formulation comprising about 5.01 g granules, about 1.32 g P407 and about 4.25 g water was produced in accordance with Table 12.

TABLE 12
Composition of putty formulation

| Granule size | | | Wt % of putty |
|---|---|---|---|
| range (μm) | | Weight (g) | formulation |
| 50 | 100 | 0.46 | |
| 100 | 200 | 0.91 | |
| 200 | 400 | 1.36 | |
| 400 | 600 | 1.14 | |
| 600 | 800 | 1.14 | |

| | | |
|---|---|---|
| Total weight of granules | 5.01 | 47% |
| Carrier components | | |
| P407 | 1.32 | 13% |
| Water | 4.25 | 40% |
| Total weight of carrier | 5.57 | 53% |
| Total weight | 10.58 | 100% |

### Example 4

A putty formulation comprising about 5.01 g granules, about 2.32 g P407 and about 4.25 g water was produced in accordance with Table 13.

TABLE 13
Composition of putty formulation

| Granule size | | | Wt % of putty |
|---|---|---|---|
| range (μm) | | Weight (g) | formulation |
| 50 | 100 | 0.46 | |
| 100 | 200 | 0.91 | |
| 200 | 400 | 1.36 | |
| 400 | 600 | 1.14 | |
| 600 | 800 | 1.14 | |

| | | |
|---|---|---|
| Total weight of granules | 5.01 | 43% |
| Carrier components | | |
| P407 | 2.32 | 20% |
| Water | 4.25 | 37% |
| Total weight of carrier | 6.57 | 57% |
| Total weight | 11.58 | 100% |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A putty formulation comprising:
granules comprising a macroporous cement composition comprising:
80-95 wt % hydroxyapatite; and
β-calcium pyrophosphate, wherein the porosity of the macroporous cement composition is 60-80 vol % as determined using Archimedes method;
a liquid carrier, and
a binder material, wherein
the average particle size of the granules is 50-800 μm as determined by sieving and/or scanning electron microscopy; and
the portion of granules in the putty formulation is 25-50 wt %.

2. The putty formulation according to claim 1, wherein the macroporous cement composition further comprises α-tricalcium phosphate.

3. The putty formulation according to claim 2, wherein the macroporous cement composition comprises:
80-95 wt % hydroxyapatite;
0.1-10 wt % β-calcium pyrophosphate; and
<10 wt % α-tricalcium phosphate.

4. The putty formulation according to claim 1, wherein the portion of granules in the putty formulation is 30-50 wt %.

5. The putty formulation according to claim 4, wherein the portion of granules in the putty formulation is 40-50 wt %.

6. The putty formulation according to claim 1, wherein the portion of binder material in the putty formulation is 5-25 wt %.

7. The putty formulation according to claim 6, wherein the portion of binder material in the putty formulation is 10-25 wt %.

8. The putty formulation according to claim 7, wherein the portion of binder material in the putty formulation is 13-25 wt %.

9. The putty formulation according to claim 1, wherein the liquid carrier is water.

10. The putty formulation according to claim 1, wherein the putty formulation comprises 30-70 wt % liquid carrier.

11. The putty formulation according to claim 10, wherein the putty formulation comprises 35-65 wt % liquid carrier.

12. The putty formulation according to claim 1, wherein the binder material comprises carboxymethyl cellulose.

13. The putty formulation according to claim 1, wherein the binder material comprises a triblock copolymer comprising a central hydrophobic block of polypropylene glycol flanked by two hydrophilic blocks of polyethylene glycol.

14. The putty formulation according to claim 13, wherein the binder material comprises poloxamer 407.

15. The putty formulation according to claim 1, further comprising an antioxidant.

16. The putty formulation according to claim 15, wherein the antioxidant is ascorbic acid.

17. The putty formulation according to claim 1, wherein the putty formulation is a sterilized putty formulation.

18. The putty formulation according to claim 1, wherein the putty formulation comprises the granules and a gel comprised of the liquid carrier and the binder material.

19. A method of manufacturing a putty formulation comprising the steps of:
obtaining granules comprising a macroporous cement composition comprising:
80-95 wt % hydroxyapatite; and
at least one bioactive calcium phosphate phase, wherein
the porosity of the granules is 60-80 vol % as determined by Archimedes method; and
the granules have an average particle size of 50-800 μm as determined by sieving and/or scanning electron microscopy; and
mixing the granules with a liquid carrier and a binder material, wherein the portion of granules in the putty formulation is 25-50 wt %.

20. The method according to claim 19, wherein mixing the granules comprises mixing the granules with a gel comprised of the liquid carrier and the binder material.

21. The method according to claim 19, wherein the at least one bioactive calcium phosphate phase comprises β-calcium pyrophosphate.

22. The method according to claim 21, wherein the at least one bioactive calcium phosphate phase comprises β-calcium pyrophosphate and α-tricalcium phosphate.

23. The method according to claim 22, wherein the macroporous cement composition comprises:
80-95 wt % hydroxyapatite;
0.1-10 wt % β-calcium pyrophosphate; and
<10 wt % α-tricalcium phosphate.

24. The method according to claim 19, wherein the step of obtaining the granules comprises:
mixing α-tricalcium phosphate, β-calcium pyrophosphate, and a sacrificial phase forming a dry powder mix, wherein all components are in solid form;
mixing the dry powder mix and a liquid forming a paste;
curing the paste at a temperature above room temperature;
terminating the chemical reaction associated with the curing after a predetermined time period selected so that a predetermined amount of α-tricalcium phosphate remains unreacted forming a solid cement composition, the predetermined amount being above 1 wt %;
leaching the sacrificial phase from the solid cement composition; and
drying at a temperature above room temperature, further to form the granules comprising the macroporous cement composition.

25. The method according to claim 19, further comprising a step of sterilizing the putty formulation.

26. The method according to claim 25, wherein the step of sterilizing the putty formulation comprises sterilizing the putty formulation using irradiation.

27.-30. (canceled)

31. A method for treating a bone defect comprising delivering a putty formulation according to claim 1 into a bone void in a subject.
